(19) United States
(12) Patent Application Publication
(10) Pub. No.: US 2009/0220963 A1
(43) Pub. Date: Sep. 3, 2009

(54) KIT SUITABLE FOR SCREENING AND ESTABLISMENT OF OPTIMAL AMPLIFICATION CONDITION IN PCR CONSTRUCTED WITH DRIED-FORMULATED PCR REAGENT AND METHOD FOR PRODUCING THE SAME

(75) Inventors: Seongjun Yoon, Seoul (KR); Sanghyeon Kang, Seoul (KR); Jisung Park, Sungnam-Si (KR); Bona Park, Sungnam-Si (KR); Jungok Kang, Seoul (KR); Mansoon Park, Sungnam-Si (KR)

Correspondence Address: Ballard Spahr Andrews & Ingersoll, LLP, SUITE 1000, 999 PEACHTREE STREET, ATLANTA, GA, 30309-3915, US

(73) Assignee: iNtRON Biotechnology, Inc., Sungnam-Si (KR)

(21) Appl. No.: 12/126,513

(22) Filed: May 23, 2008

(30) Foreign Application Priority Data
Feb. 28, 2008 (KR) 10-2008-0018435

(51) Int. Cl. C12Q 1/68

(52) U.S. Cl. 435/6

(57) ABSTRACT

The present invention relates to a kit for screening and establishing optimal amplification condition for individual PCR using a dried-formulated PCR reagent which is composed of different combinations of various components affecting PCR result, to provide a method for screening and establishment of optimal amplification in PCR constructed with a dried-formulated PCR reagent. According to the present invention, researchers can perform PCR under the optimal amplification conditions appropriate for individual PCR even with a PCR-related product constructed from a dried-formulated PCR reagent, suggesting that a unique target gene can be efficiently amplified by PCR constructed with a dried-formulated PCR reagent under the more appropriate conditions.

### CROSS REFERENCE TO RELATED APPLICATIONS

This application claims priority to Korean Patent Application No. 10-2008-0018435, filed Feb. 28, 2008, which application is incorporated by this reference in its entirety.

### TECHNICAL FIELD

The present invention relates to a kit suitable for screening and establishment of optimal amplification condition in PCR constructed with a dried-formulated PCR reagent, and a method for producing the same. Precisely, the present invention relates to a screening kit for reaction conditions of PCR constructed with a dried-formulated PCR reagent which is composed of different combinations of factors with various concentrations affecting PCR result and is suitable for screening and establishment of optimal PCR reaction condition, and a method for producing the same.

### BACKGROUND ART

PCR is a molecular biological method that is capable of amplifying a target DNA exponentially. Any part of DNA can be amplified once its sequence is identified. PCR was first proposed by K. Mullis in mid-1980s. Since then, PCR has been widely used in biological research fields including molecular genetics which studies genes. PCR exploits the DNA replication activity of DNA polymerase. DNA polymerase facilitates the synthesis of complementary DNA molecule by using single stranded DNA molecule as a template. This single stranded DNA molecule can be simply obtained by boiling a double stranded DNA molecule. This procedure is called ‘DNA denaturation’. In order for DNA polymerase to start DNA synthesis, start site has to be double stranded DNA form. So, to form double stranded DNA, small DNA fragments capable of binding complementarily to both ends of a template DNA should be added in PCR. This complexiation process between DNA fragments and a template DNA is annealing. Only after annealing, DNA synthesis by DNA polymerase can be started. The complementary DNA fragments capable of binding to both ends of a target DNA sequence to be amplified are called oligonucleotide primer or simply primer. After binding of the primer to the template DNA, DNA synthesis extends to the other end by DNA polymerase. PCR cycle is generally consisted of the following steps:

1) Denaturation which changes double-stranded template DNA molecule into single-stranded DNA molecule;

2) Annealing of the primer to the single-stranded DNA template; and

3) Elongation which synthesizes a DNA molecule complementary to the template DNA by DNA polymerase.

After completion of the first PCR cycle, the original template DNA and the PCR product are both used as DNA templates in the subsequent PCR cycle. So, as PCR cycle is repeated, the number of DNA templates is increasing. In an idealized case, the number of existing DNA molecules in a PCR is 2ⁿ after n cycles. As a result, (2ⁿ−1) copies of the original template DNA are synthesized. In PCR cycles, the first step is the template denaturation step. The template denaturation step requires high temperature of at least 90° C. In this step, DNA polymerase may be denatured. The DNA polymerases initially employed had low thermo-stability which is called mesophilic DNA polymerase. In this case with mesophilic DNA polymerase, fresh DNA polymerase had to be added to the PCR reaction mixture in each PCR cycle. However, since a thermo-stable DNA polymerase was found in Thermus aquaticus, a thermophilie living in hot spring, the addition of fresh DNA polymerase to PCR reaction mixture in each PCR cycle has not been necessary and DNA polymerase is added just once when PCR is started. The optimal temperature for this kind of thermo-stable DNA polymerase (Taq DNA polymerase) is 72° C. and it is still stable at 94° C. The discovery of the thermo-stable Taq DNA polymerase facilitated PCR and paved a way for PCR to be used in various research fields (Science 252: 1643-1651, 1991). So now, PCR is acknowledged as a powerful technique used in various research fields.

Since the discovery of the thermo-stable Taq DNA polymerase, PCR techniques have been astonishingly advanced mainly by the discovery of novel DNA polymerases and the development of novel PCR techniques. Newly discovered or developed DNA polymerases are Tth DNA polymerase (from Thermus thermophilus), Tfl DNA polymerase (from Thermus flavus), Hot Tub DNA polymerase (from Thermus ubiquitos), Ultma DNA polymerase (from Thermotoga maritima), Pfu DNA polymerase (from Pyrococcus furiosus), Vent DNA polymerase (from Thermococcus litoralis) and Tli DNA polymerase (from Thermococcus litoralis) and Pwo DNA polymerase (from Pyrococcus woesei). The forgoing DNA polymerases are all brand name products. Because these DNA polymerases are distinguished one another in their characteristics, they have been utilized in different PCRs according to their unique properties. Precisely, they are different in DNA synthesizing speed, the number of nucleotides synthesized from the binding of the polymerase to a template DNA to the separation, preference to the kinds of template-primer, and sensitivity to inhibitory materials. Recently, a method has been developed to use at least two of these DNA polymerases together. Using this blend of different DNA polymerases (i.e., mixed DNA polymerases) is expected to have advantages because merits of both or multiple DNA polymerases can be all utilized or the overall inhibitory effect by an inhibitor can be reduced.

PCR techniques developed so far are as follows: rapid PCR characterized by reduced time for amplification; direct PCR capable of direct using of unpurified samples; reverse transcriptase-PCR (RT-PCR) which combines reverse transcription with PCR and thereby can use RNA molecule as a template; hot-start PCR with improved PCR specificity by reducing the non-specific amplification occurring at room temperature; and real-time PCR facilitating real-time monitoring of PCR reaction. In addition, many techniques and methods have been developed but detailed explanations on these are not given herein.

Along with the development of novel DNA polymerases and the advancement of PCR techniques, studies to perform PCR more easily have been undergoing. There have been various attempts to facilitate PCR. The first attempt was to use master mixture (master mix; pre-mixture; premix) in set-up PCR reaction. To set-up PCR reaction, each component necessary for PCR is mixed together in a single tube to prepare PCR reaction mixture. If a master mixture, which is the mixture where each and every PCR component is mixed at a desired concentration, is used in preparing PCR reaction mixture, it will be much easier and simpler. When such a master mixture is used in preparing PCR reaction mixture, the only thing to do is to add a template, primers and water to the forgoing master mixture. By this, pipetting necessary for preparing PCR reaction mixture can be reduced greatly and deviations among PCR reactions can also be reduced. Accordingly, the use of a master mixture can reduce carry-over contamination accompanied by repeated pipetting. The early master mixture was prepared by simple mixing of each component taken from its stock solution, so that it was a solution type (i.e., aqueous solution).

In the solution type master mixture, degrees of freedom of each component are high so that deactivation (or inactivation) keeps going on, suggesting that PCR-related products containing the solution type master mixture are unstable during delivery and storage. Needless to say, the low stability of product makes matter worse in delivery and storage, asking additional efforts and causing great inconvenience. To overcome these problems, stability of the master mixture has to be improved. And thus, a dried-formulated master mixture has been developed (Korean Patent No. 0730364). In present invention, the dried-formulated PCR master mixture prepared by drying process such as freeze-drying, drying at elevated temperature, drying at room temperature, vacuum drying, etc, is indicated as one of those terms, “dried-formulated PCR master mixture”, “dried-formulated master mixture for PCR”, “dried-formulated master mixture”, “dried-formulated PCR reagent”, “dried-formulated reagent for PCR”, or “dried-formulated reagent”. Besides serving convenience in use, the use of PCR-related product containing such dried-formulated PCR master mixture gave additional advantages such as convenience in delivery and storage due to improved stability of the PCR-related product attributed to the improved stability of master mixture. These PCR-related products containing the dried-formulated PCR master mixture can be applied effectively in every type PCRs and particularly effective in the field requiring repeated PCR, for example diagnosis including genotyping and disease diagnosis. Largely, they are effective in PCRs performed in clinical fields and environmental fields.

However, unlike the solution type PCR reagents, such dried-formulated PCR reagents are provided by a manufacturer with compositions fixed, so that it is very difficult to change reaction conditions of experimenter's own account. The changeable factors by experimenter are those related to a primer such as the kind of primer (i.e., primer sequence), GC content of primer, Tm of primer, and concentration of primer applied in PCR; those related to PCR operation conditions such as the time period for each PCR step, number of PCR cycle and temperature of each PCR step. Besides aforementioned factors, there are many factors affecting the result of PCR, for example, magnesium ion concentration, the kind and concentration of DNA polymerase, composition of reaction buffer, etc. Despite these factors are important, an experimenter could not be able to adjust those factors with PCR-related products containing the dried-formulated PCR reagent. So, it is required to develop a method and kit applicable in screening and determining optimal PCR reaction conditions for individual PCR with the dried-formulated PCR reagent.

The present inventors completed this invention by providing a method for screening PCR conditions appropriate for individual PCR using dried-formulated PCR reagents and a kit for the same.

The description of the present invention referred to research papers and patent descriptions and the citation is marked in parentheses. Not a part of the papers and patent descriptions but the entire of them are enclosed as references so that the techniques and details of the present invention can be illustrated more clearly.

Disclosure

### DETAILED DESCRIPTION

Description of Drawings

The application of the preferred embodiments of the present invention is best understood with reference to the accompanying drawings, wherein:

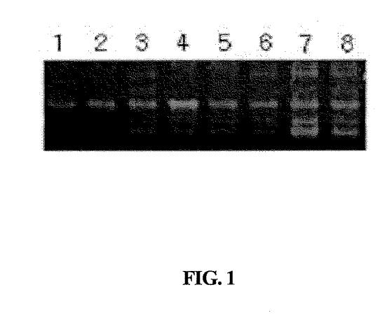

FIG. 1 is a photograph of agarose gel electrophoresis presented as a representative example. The intensity of the corresponding band was measured on the gel by a GS-800 Calibrated Densitometer, followed by investigation of the amount of amplified PCR product and specificity.

### TECHNICAL PROBLEM

It is an object of the present invention to solve the problems of the conventional techniques and methods and to provide a solution for them.

Thus, the present inventors provide a method for screening PCR reaction conditions suitable for individual PCR using a dried-formulated PCR reagent and a kit facilitating thereof.

### TECHNICAL SOLUTION

To achieve the above object, the present invention provides a method for screening and establishment of optimal PCR reaction conditions appropriate for individual PCR using a dried-formulated PCR reagent and for which the present invention provides a kit containing a dried-formulated reagent with various combinations of core components and additional components.

The result of PCR depends on components and conditions applied in PCR. Therefore, to amplify a target gene by PCR, appropriate PCR reaction conditions for the unique target gene amplification have to be determined first. Routinely, to determine the optimal PCR reaction condition, multiple PCRs need to be performed with different PCR reaction conditions. But, the PCR-related product containing the dried-formulated PCR reagent are already set at compositions and components selected by manufacturer, so that it is difficult, in fact impossible, to adjust and modify PCR reaction conditions of experimenter's own account. So, it has been requested the compositions and components of the dried-formulated PCR reagent can be varied for screening of proper PCR reaction conditions for individual PCR. That is, experimenters want to establish their own PCR reaction conditions suitable for their purpose by screening PCR reaction conditions using a dried-formulated master mixture.

The present inventors tried to develop a method to meet the above request. The present inventors finally completed this invention by providing a starter kit which is a kind of screening kit for PCR reaction conditions using a dried-formulated master mixture and avoids multiple PCRs for screening PCR reaction conditions.

The starter kit is suitable for high throughput screening (HTS) for establishing PCR reaction conditions appropriate for individual PCR. To screen proper PCR reaction conditions fast and easily, this kit facilitates screening of optimal conditions for individual PCR using a dried-formulated PCR reagent being provided with various combinations of important factors such as those affecting the result of PCR including composition for reaction buffer, magnesium ion concentration and DNA-helix-destabilization-related additive which can improve PCR efficiency with high GC contented template; the type of DNA polymerase and the concentration of DNA polymerase. With this kit, appropriate PCR reaction conditions can be screened by changing the kind of primer, primer concentration, the time period for PCR step, number of PCR cycles and temperature for PCR step. Basically, this kit of the present invention enables PCR constructed with a dried-formulated reagent under the proper PCR reaction conditions determined according to the method disclosed in the present invention. The kit of the present invention can be further applied in the development of a diagnostic kit. It has been very difficult for an experimenter to develop a diagnostic kit using a dried-formulated PCR reagent due to its prefixed composition. And it is our goal to overcome the said problem.

In this invention, indispensible components which are necessary for PCR are called “core component”, “core factor”, “necessary component” or “necessary factor” and other components added to improve PCR efficiency, in addition to the above necessary components, are called “additional component” or “additional factor”.

To promote understanding, the core factors are limited to magnesium ion, buffering component of reaction buffer, monovalent ion of reaction buffer and DNA polymerase, and other components are all classified into the additional factors.

In a preferred embodiment of the present invention, the concentration of magnesium ion was varied in the range of 1.5 mM-3.5 mM in steps of 0.2 mM or 0.5 mM. The concentration range and step can be varied.

The buffering component of the reaction buffer herein is exemplified by Tris, Tricine and Hepes (N-[2-hydroxyethyl]piperazine-N′-[2-ethanesulfonic acid]), but not always limited thereto.

The monovalent ion of the reaction buffer is exemplified by ammonium ion, potassium ion and sodium ion, but not always limited thereto.

The DNA polymerase herein is exemplified by Taq DNA polymerase, Tth DNA polymerase, Tfl DNA polymerase, Hot Tub DNA polymerase, Ultma DNA polymerase, Pfu DNA polymerase, Vent DNA polymerase, Tli DNA polymerase and Pwo DNA polymerase (These are all brand name products), but not always limited thereto.

The additional factors include DNA-helix-destabilization-related materials added to improve amplification efficiency of PCR using a high GC contented template or primer and PCR enhancers, etc.

The DNA-helix-destabilization-related materials are exemplified by betaine (Biochemistry 32: 137-144, 1993), tetraalkylammonium (J. Mol. Biol. 86: 469-489, 1974), proline (FEBS Letters 410: 201-205, 1997), glycerol (Nucleic Acids Res. 27: 1566-1568, 1999), ethylene glycol (Nucleic Acids Res. 27: 1566-1568, 1999), etc, but not always limited thereto.

PCR enhancers are exemplified by carbohydrates such as sucrose and trehalose capable of increasing PCR efficiency by regulating osmolarity; gelatin; and polyethylene glycol (PEG), but not always limited thereto.

The present invention provides a method for screening and establishing optimal reaction conditions of PCR constructed with a dried-formulated PCR reagent as HTS format and a kit facilitating the same.

The method and the kit of the present invention include the factors affecting the result of PCR, which can be one or more factors. The factor included in the kit of the present invention can be a kind of specific component or a concentration of the specific component.

The PCR to be screened by the kit and method of the present invention can be RT-PCR using DNA polymerase as well as PCR.

The construction form of the kit using the dried-formulated PCR reagent of the present invention is as follows, but not always limited thereto.

(1) tube #1-tube #8 of 8-strip containing the same PCR reaction mixture but different concentrations of magnesium ion;

(2) tube #1-tube #8 of 8-strip containing the same PCR reaction mixture but different concentrations of DNA-helix-destabilization-related material for PCR using a template or a primer having high GC content;

(3) tube #1-tube #8 of 8-strip containing the same PCR reaction mixture but different amounts of DNA polymerase;

(4) tube #1-tube #8 of 8-strip containing the same PCR reaction mixture but different kinds of DNA polymerase; and

(5) tube #1-tube #8 of 8-strip containing the same PCR reaction mixture but different compositions of reaction buffer.

The above is only an example and can be modified, that is:

(1) Instead of 8-strip, it can be 96-strip or more or less. The differences can be 8 or 4, or can vary more than 8 or less than 4 differences. That is, 8 differences are only an example.

(2) The reaction mixture can be formulated as a dried-formulated master mixture by freeze-drying, drying at elevated temperature, drying at room temperature and vacuum drying.

(3) DNA polymerase can be selected from the group consisting of Taq DNA polymerase, Pfu DNA polymerase, DNA polymerase for Hot-Start, DNA polymerase for Long PCR, Tth DNA polymerase, Tfl DNA polymerase, Hot Tub DNA polymerase, Ultma DNA polymerase, Vent DNA polymerase, Tli DNA polymerase and Pwo DNA polymerase, but not always limited thereto.

(4) The kit can be applied not only in PCR but also in RT-PCR. That is, the kit can be useful for establishing reaction conditions for every PCR.

So, by adjusting or changing the kind of a primer, the concentration of a primer, the time period for PCR, the number of PCR cycles, the temperature of PCR step, optimal PCR conditions suitable for individual PCR and appropriate PCR-related product for individual amplification can be selected.

With the kit and method of the present invention, researchers can screen optimal PCR reaction conditions as HTS format for PCR constructed with a dried-formulated PCR reagent and thereafter perform PCR under proper PCR reaction conditions with the PCR-related product containing a dried-formulated PCR reagent. According to the present invention, proper PCR reaction conditions to individual PCR can be screened with the kit containing a dried-formulated PCR reagent. Again, in spite of using a PCR-related product constructed with a dried-formulated PCR reagent, efficient PCR under the optimized condition for amplification of their unique target gene is possible. So, researchers can decide PCR reaction conditions appropriate for their own amplifications simply by one-time PCR using the kit of the present invention. The PCR-related product containing a dried-formulated PCR reagent can be applied effectively in every type PCRs and particularly effective in the field requiring repeated PCR, for example diagnosis including genotyping and disease diagnosis. In a wide sense, the PCR-related product containing the dried-formulated PCR reagent is effective in PCRs performed in clinical field, food hygiene field, and environmental field, too. So, it is the effect of the present invention to facilitate establishment or setting up of PCR reaction conditions of their own account by using a dried-formulated PCR reagent, which has been not possible so far. This advantage is especially effective in the development of a diagnostic kit or product. It has been very difficult up to date for a researcher to perform efficient PCR under proper PCR reaction condition using a dried-formulated PCR reagent. The present invention overcomes this disadvantage and is effective in the development of a diagnostic kit or product.

Practical and presently preferred embodiments of the present invention are illustrative as shown in the following Examples.

However, it will be appreciated that those skilled in the art, on consideration of this disclosure, may make modifications and improvements within the spirit and scope of the present invention.

The following embodiments illustrate some of major factors and points of the present invention, but these cannot limit the spirit and scope of the present invention but can be applied in various PCR components. In a preferred embodiment of the present invention, the kit is composed of 8-strip but this is only an example and the kit can also be composed of 96 strip or others. The container herein is PCR tube but can be microplate or another container. In a preferred embodiment of the present invention, concentrations are varied as 8 different levels, but they can be 4 different levels and more than 8 or less than 4 levels can also be accepted. So, 8 different levels of the concentration are simply an example. In another preferred embodiment of the present invention, screening of optimal concentration of one or two components is illustrated, but screening of optimal concentrations of three or more is also possible. For preparing a dried-formulated PCR master mixture, drying at elevated temperature was performed in this invention, but any drying method such as freeze-drying, drying at elevated temperature, drying at room temperature and vacuum drying can be used. In a preferred embodiment of the present invention, PCR was illustrated as an applicable example, but reverse transcription-PCR using RNA as a template, which is the combined method of reverse transcription and PCR, can be another example. At this time, only the components supporting reverse transcription are necessarily added. This, however, is well known to those in the art, so that explanation is not given.

In Example 1, screening of optimal concentrations of core components for PCR is illustrated and at this time magnesium ion was selected as a target component. However, screening of optimal concentration of magnesium ion is just an example and the method of the present invention can be further applied in screening of concentrations of DNA polymerase and components of reaction buffer. In addition, lots of general necessary components for PCR can be application targets. As shown in Example 2, many effective additional components, in addition to the core components, can be application targets of the present invention.

### EXAMPLE 1

### Kit for Screening Optimal Concentration of Magnesium Ion

In this example, screening of optimal concentration of a core component for PCR was performed.

The kit for screening optimal concentration of magnesium ion which might have serious effect on the result of PCR was prepared as follows and then optimal concentration of magnesium ion was determined. Magnesium chloride (MgCl₂) was added to tube #1-tube #8 of 8-strip containing the same PCR reaction mixture at different concentrations. According to the method described in Korean Patent No. 0730364, the above mixture was dried by drying at elevated temperature to give a dried-formulated PCR master mixture. The composition of the same PCR reaction mixture used is as follows and is called ‘basic composition’: 30 mM Tris-HCl (pH 9.0), 15 mM KCl, 15 mM NaCl, 5 units Taq DNA polymerase, 30 mM trehalose and 0.005% (w/v) xylene cyanol. This composition can be adjusted and the present invention is not limited by the basic composition. Magnesium chloride was added to tube #1-tube #8 having the same basic composition at different concentrations of 1.5 mM, 1.8 mM, 2.0 mM, 2.3 mM, 2.5 mM, 2.8 mM, 3.0 mM and 3.5 mM respectively. The prepared aqueous solution was dried to give a dried-formulated reagent.

The template used for PCR in this example was human gDNA (genomic DNA) extracted from K562, a human cell-line. Extraction of the gDNA was performed by using a G-spin™ Genomic DNA Extraction kit (for Cell/Tissue) according to the manufacturer's instruction (iNtRON Biotechnology). The target gene for amplification by PCR in this example was 1.8 kbp sized beta-globin fragment. The NCBI accession number of the beta-globin gene is NW925006.1. The amount of the template DNA was 4 ng. Primers used for PCR herein were the forward primer 5′-GAA GGC TCA TGG CAA GAA AG-3′ (SEQ. ID. NO: 1) and the reverse primer 5′-GAT TCC GGG TCA CTG TGA GT-3′ (SEQ. ID. NO: 2). PCR was performed by using a thermal cycler as follows; initial denaturation at 94° C. for 2 minutes, denaturation at 94° C. for 20 seconds, annealing at 60° C. for 20 seconds, polymerization at 72° C. for 2 minutes, 35 cycles from denaturation to polymerization, final extension at 72° C. for 2 minutes and the reactant stood at 4° C.

After PCR, the PCR reaction mixture was subjected to electrophoresis on 1% agarose gel. After electrophoresis, the gel was examined by using a GS-800 Calibrated Densitometer (Bio-Rad) to measure the amount of amplicon of the target gene and to investigate the specificity. The specificity herein indicates the ratio of the amplification of a target gene to the non-specific amplification. So, high specificity means the amplification of a target gene is dominant. In this example, the target gene is approximately 1.8 kbp in size. And it was confirmed that 1.8 kbp sized PCR product was successfully amplified. FIG. 1 is a photograph of electrophoresed gel and the result is presented in Table 1. In Table 1, the amount of amplicon of the target gene is presented by “+”. If amplification is done successfully, it gets more “+”. And in examination of specificity, more “+” indicates the amplification of a target gene is more dominant than non-specific amplification.

TABLE 1

| | Tube | Tube | Tube | | Tube | Tube | Tube | Tube |
|---|---|---|---|---|---|---|---|---|
| | 1 | 2 | 3 | Tube 4 | 5 | 6 | 7 | 8 |
| Conc. of | 1.5 | 1.8 | 2.0 | 2.3 | 2.5 | 2.8 | 3.0 | 3.5 |
| magnesium | | | | | | | | |
| (mM) | | | | | | | | |
| Ampli- | + | ++ | +++ | +++++ | ++++ | ++++ | ++++ | ++++ |
| fication | | | | | | | | |
| yield | | | | | | | | |
| Specificity | ++++ | ++++ | +++ | +++++ | +++ | ++ | + | + |
| Selection | | | | Selected | | | | |

As shown in the above results, optimal concentration of magnesium ion in this case could be determined by one-time PCR.

### EXAMPLE 2

### Kit for Screening Optimal Concentration of DNA-Helix-Destabilization-Related Material

Screening of optimal concentration of an additional component which is not a core component for PCR but is an effective ingredient was performed in this example.

As an example of an additional component that might affect the result of PCR, DAN-helix-destabilization-related material was selected. Then, the kit for screening the optimal concentration of the DNA-helix-destabilization-related material was prepared and used to determine the optimal concentration thereof. In this example, betaine was selected as the DNA-helix-destabilization-related material.

Experiment was performed as follows. Betaine was added to tube #1-tube #8 of 8-strip containing the same PCR reaction mixture at different concentrations. According to the method described in Korean Patent No. 0730364, the above mixture was dried by drying at elevated temperature to give a dried-formulated PCR master mixture. The basic composition of the same PCR reaction mixture used is as follows: 30 mM Tris-HCl (pH 9.0), 15 mM KCl, 15 mM NaCl, 2 mM magnesium chloride, 5 units Taq DNA polymerase, 30 mM trehalose and 0.005% (w/v) xylene cyanol. This basic composition can be adjusted and the present invention is not limited by the basic composition. Betaine was added to tube #1-tube #8 having the same basic composition at different concentrations of 0.25 M, 0.5 M, 0.75 M, 1.0 M, 1.25 M, 1.5 M, 1.75 M and 2.0 M respectively. The prepared aqueous solution was dried to give a dried-formulated reagent.

The template used for PCR in this example was human gDNA extracted from K562, a human cell-line, so was in Example 1. Extraction of the gDNA was performed by using a G-spin™ Genomic DNA Extraction kit (for Cell/Tissue) according to the manufacturer's instruction (iNtRON Biotechnology). The target gene for amplification in this example was different from that of Example 1. In Example 2, the target gene was 196 bp sized human retinoblastoma 1 (RB-1) gene fragment. The NCBI accession number of the RB-1 gene is NW925473.1. The amount of the template DNA was 10 ng. Primers used for PCR herein were the forward primer 5′-CAG GAC AGC GGC CCG GAG-3′ (SEQ. ID. NO: 3) and the reverse primer 5′-CTG CAG ACG CTC CGC CGT-3′ (SEQ. ID. NO: 4). PCR was performed as follows by using a thermal cycler; initial denaturation at 94° C. for 2 minutes, denaturation at 94° C. for 30 seconds, annealing at 63° C. for 35 seconds, polymerization at 72° C. for 40 seconds, 30 cycles from denaturation to polymerization, final extension at 72° C. for 2 minutes and the reactant stood at 4° C.

After PCR, the PCR reaction mixture was applied to 1% agarose gel for electrophoresis. After electrophoresis, the gel was examined by using a GS-800 Calibrated Densitometer (Bio-lad) to measure the amount of amplicon of the target gene and to investigate specificity. As a result, 196 bp sized PCR product was successfully amplified and the result is presented in Table 2. In Table 2, amplification yield is presented by the same manner as presented in Table 1. That is, the relative value of the intensity of the corresponding band is presented as “+”. The more “+” indicates the higher yield of amplification. In the investigation of specificity, the more “+” indicates the amplification of the target gene is more dominant than non-specific amplification.

TABLE 2

| | Tube | Tube | Tube | | Tube | Tube | Tube | Tube |
|---|---|---|---|---|---|---|---|---|
| | 1 | 2 | 3 | Tube 4 | 5 | 6 | 7 | 8 |
| Conc. of | 0.25 | 0.5 | 0.75 | 1.0 | 1.25 | 1.5 | 1.75 | 2.0 |
| betaine | | | | | | | | |
| (M) | | | | | | | | |
| Ampli- | +++ | ++++ | ++++ | +++++ | +++++ | ++++ | ++++ | ++++ |
| fication | | | | | | | | |
| yield | | | | | | | | |
| Specificity | +++ | ++++ | ++++ | +++++ | ++++ | ++++ | ++++ | +++ |
| Selection | | | | Selected | | | | |

As shown in the above results, optimal concentration of betaine (1.0 M) in this case could be determined by one-time PCR.

### EXAMPLE 3

### Kit for Simultaneous Screening of Optimal Concentrations of Magnesium and DNA-Helix-Destabilization-Related Material

In this example, simultaneous screening of optimal concentrations of one of core components and one of additional components for PCR was performed. The kit of this example facilitated simultaneous screening of various components regardless of core components or additional components. And, the target components for simultaneous screening were not necessarily one of core components and one of additional components. In this example, simultaneous screening of two components is illustrated as an example but not limited thereto and more than two components can be applied as well.

In this example, the kit for simultaneous screening of optimal concentrations of magnesium ion and betaine was constructed, with which optimal concentrations of both magnesium ion and betaine were simultaneously determined.

Experiment was performed as follows. 8 combinations of 4 different magnesium chloride concentrations and 2 different betaine concentrations were added to tube #1-tube #8 of 8-strip containing the same PCR reaction mixture. The basic composition of the same PCR reaction mixture used is as follows: 30 mM Tris-HCl (pH 9.0), 15 mM KCl, 15 mM NaCl, 5 units Taq DNA polymerase, 30 mM trehalose and 0.005% (w/v) xylene cyanol. This basic composition can be adjusted and the present invention is not limited by the basic composition. Magnesium chloride and betaine were added to tube #1-tube #8 having the same basic composition as presented in Table 3.

TABLE 3

| | Tube | Tube | Tube | Tube | Tube | Tube | Tube | |
|---|---|---|---|---|---|---|---|---|
| | 1 | 2 | 3 | 4 | 5 | 6 | 7 | Tube 8 |
| Conc. of | 1.8 | 2.0 | 2.3 | 2.5 | 1.8 | 2.0 | 2.3 | 2.5 |
| magnesium | | | | | | | | |
| (mM) | | | | | | | | |
| Conc. of | 0.5 | 0.5 | 0.5 | 0.5 | 1.0 | 1.0 | 1.0 | 1.0 |
| betaine (M) | | | | | | | | |

According to the method described in Korean Patent No. 0730364, the above mixture was dried by drying at elevated temperature as described in Example 1 and Example 2 to give a dried-formulated PCR master mixture.

The template used for PCR in this example was human gDNA extracted from K562, a human cell-line. Extraction of the gDNA was performed by using a G-spin™ Genomic DNA Extraction kit (for Cell/Tissue) according to the manufacturer's instruction (iNtRON Biotechnology). In this example, the target gene was 1.2 kbp sized human c-jun gene fragment. The NCBI accession number of the c-jun gene is NW921351.1. The amount of the template DNA was 10 ng. Primers used for PCR herein were the forward primer 5′-GGG AGG GGA CCG GGG AAC AGA G-3′ (SEQ. ID. NO: 5) and the reverse primer 5′-GAA CAG TCC GTC ACT TCA CGT G-3′ (SEQ. ID. NO: 6). PCR was performed as follows by using a thermal cycler; initial denaturation at 94° C. for 2 minutes, denaturation at 94° C. for 30 seconds, annealing at 66° C. for 35 seconds, polymerization at 72° C. for 1 minute, 30 cycles from denaturation to polymerization, final extension at 72° C. for 2 minutes and the reactant stood at 4° C.

After PCR, the PCR reaction mixture was applied to 1% agarose gel for electrophoresis. After electrophoresis, the gel was examined by using a GS-800 Calibrated Densitometer (Bio-lad) to measure the amount of amplicon of the target gene and to investigate specificity. As a result, 1.2 kbp sized PCR product was successfully amplified and the result is presented in Table 4. In Table 4, amplification yield is presented. That is, the relative value of the intensity of the corresponding band is presented as “+”. The more “+” indicates the higher yield of amplification. In the investigation of specificity, the more “+” indicates the amplification of the target gene is more dominant than non-specific amplification.

TABLE 4

| | Tube | Tube | Tube | Tube | Tube | | Tube | Tube |
|---|---|---|---|---|---|---|---|---|
| | 1 | 2 | 3 | 4 | 5 | Tube 6 | 7 | 8 |
| Conc. of | 1.8 | 2.0 | 2.3 | 2.5 | 1.8 | 2.0 | 2.3 | 2.5 |
| magnesium | | | | | | | | |
| (mM) | | | | | | | | |
| Conc. of | 0.5 | 0.5 | 0.5 | 0.5 | 1.0 | 1.0 | 1.0 | 1.0 |
| betaine (M) | | | | | | | | |
| Ampli- | +++ | ++++ | +++ | +++ | +++ | ++++ | ++++ | ++++ |
| fication | | | | | | | | |
| yield | | | | | | | | |
| Specificity | +++ | +++ | +++ | ++ | +++ | ++++ | +++ | ++ |
| Selection | | | | | | Selected | | |

As shown in the above result, optimal concentrations of magnesium ion (2.0 mM) and betaine (1.0 M) in this case could be determined by one-time PCR.

### EXAMPLE 4

### Kit for Screening a Proper Kind of DNA Polymerase

Screening of a proper kind of DNA polymerase, one of core components for PCR, was performed in this example. This example demonstrated that the kit of the present invention is not only useful for screening optimal concentration of a factor for PCR but also useful for screening a proper kind of a factor.

Screening of a proper kind of DNA polymerase was performed as follows. Different kinds of DNA polymerases were added to tube #1-tube #8 of 8-strip containing the same PCR reaction mixture, respectively. According to the method described in Korean Patent No. 0730364, the above mixture was dried by drying at elevated temperature to give a dried-formulated PCR master mixture. The basic composition of the same PCR reaction mixture used is as follows: 30 mM Tris-HCl (pH 9.0), 15 mM KCl, 15 mM NaCl, 2 mM magnesium chloride, 30 mM trehalose and 0.005% (w/v) xylene cyanol. This basic composition can be adjusted and the present invention is not limited by the basic composition. Different kinds of DNA polymerases were added to tube #1-tube #8 having the same basic composition and the total amount of the DNA polymerase(s) added was 5 units in each tube. Taq DNApolymerase was added to tube #1, Pfu DNApolymerase was added to tube #2, Vent DNA polymerase was added to tube #3, the blend of Taq DNA polymerase and Pfu DNA polymerase (10:1; by unit ratio) was added to tube #4, the blend of Taq DNA polymerase and Pfu DNA polymerase (15:1; by unit ratio) was added to tube #5, the blend of Taq DNA polymerase and Pfu DNA polymerase (20:1; by unit ratio) was added to tube #6, the blend of Taq DNA polymerase and Pfu DNA polymerase (25:1; by unit ratio) was added to tube #7, and the blend of Taq DNA polymerase and Pfu DNA polymerase (30:1; by unit ratio) was added to tube #8. The prepared aqueous solution was dried to give a dried-formulated reagent.

The template used for PCR in this example was human gDNA extracted from K562, a human cell-line, so was in Example 1. Extraction of the GDNA was performed by using a G-spin™ Genomic DNA Extraction kit (for Cell/Tissue) according to the manufacturer's instruction (iNtRON Biotechnology). The target gene for amplification in this example was different from that of Example 1. In Example 4, the target gene was 2.1 kbp sized p53 gene fragment. The NCBI accession number of the p53 gene is NW926584.1. The amount of the template DNA was 20 ng. Primers used for PCR herein were the forward primer 5′-TGC CGT CCC AAG CAA TGG AT-3′ (SEQ. ID. NO: 7) and the reverse primer 5′-TGT GCA GGG TGG CAA GTG GC-3′ (SEQ. ID. NO: 8). PCR was performed as follows by using a thermal cycler; initial denaturation at 94° C. for 2 minutes, denaturation at 94° C. for 20 seconds, annealing at 64° C. for 20 seconds, polymerization at 72° C. for 2 minutes, 35 cycles from denaturation to polymerization, final extension at 72° C. for 2 minutes and the reactant stood at 4° C.

After PCR, the PCR reaction mixture was applied to 1% agarose gel for electrophoresis. After electrophoresis, the gel was examined by using a GS-800 Calibrated Densitometer (Bio-lad) to measure the amount of amplicon of the target gene and to investigate specificity. As a result, it was confirmed that 2.1 kbp sized PCR product was successfully amplified and the result is presented in Table 5. Amplification yield is presented in Table 5 and the relative value of the intensity of the corresponding band is presented as “+”. The more “+” indicates the higher yield of amplification. In the investigation of specificity, the more “+” indicates the amplification of the target gene is more dominant than non-specific amplification.

TABLE 5

| | Tube 1 | Tube 2 | Tube 3 | Tube 4 | Tube 5 | Tube 6 | Tube 7 | Tube 8 |
|---|---|---|---|---|---|---|---|---|
| | DNA polymerase | | | | | | | |
| | | | | Taq | Taq | Taq | Taq | Taq |
| | | | | DNA | DNA | DNA | DNA | DNA |
| | | | | polymerase + | polymerase + | polymerase + | polymerase + | polymer- |
| | | | | Pfu | Pfu | Pfu | Pfu | ase + Pfu |
| | Taq | | Vent | DNA | DNA | DNA | DNA | DNA |
| | DNA | Pfu DNA | DNA | polymerase | polymerase | polymerase | polymerase | polymer- |
| | polymerase | polymerase | polymerase | (10:1) | (15:1) | (20:1) | (25:1) | ase (30:1) |
| Amplification | +++++ | +++ | ++++ | ++++ | +++++ | +++++ | +++++ | +++++ |
| yield | | | | | | | | |
| Specificity | +++ | +++++ | ++++ | ++++ | +++++ | ++++ | ++++ | +++ |
| Selection | | | | | Selected | | | |

As shown in the above, a proper kind of DNA polymerase in this case could be determined by one-time PCR.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A combinatorial PCR screening kit for screening and determining optimal PCR conditions, comprising a series of dried-formulated PCR reagents differing in the amount or kind of components affecting the result of PCR.

2. The combinatorial PCR screening kit for screening and determining optimal PCR conditions according to claim 1, wherein the said components affecting the result of PCR are one or more components selected from the group consisting of a magnesium ion, a DNA polymerase, a buffering component of a reaction buffer, a monovalent ion of a reaction buffer, and a PCR enhancer.

3. The combinatorial PCR screening kit for screening and determining optimal PCR conditions according to claim 2, wherein the concentration of magnesium ion is in the range of 0.5-10 mM.

4. The combinatorial PCR screening kit for screening and determining optimal PCR conditions according to claim 2, wherein the buffering component of the reaction buffer is selected from the group consisting of Tris, Tricine, and Hepes (N-[2-hydroxyethyl]piperazine-N′-[2-ethanesulfonic acid]).

5. The combinatorial PCR screening kit for screening and determining optimal PCR conditions according to claim 2, wherein the monovalent ion of the reaction buffer is selected from the group consisting of ammonium ion, potassium ion, and sodium ion.

6. The combinatorial PCR screening kit for screening and determining optimal PCR conditions according to claim 2, wherein the PCR enhancer is DNA-helix-destabilization-related material.

7. The combinatorial PCR screening kit for screening and determining optimal PCR conditions according to claim 6, wherein the DNA-helix-destabilization-related material is selected from the group consisting of betaine, tetraalkylammonium, proline, glycerol, and ethylene glycol.

8. The combinatorial PCR screening kit for screening and determining optimal PCR conditions according to claim 2, wherein the DNA polymerase is selected from the group consisting of Taq DNA polymerase, Tth DNA polymerase, Tfl DNA polymerase, Hot Tub DNA polymerase, Ultma DNA polymerase, Pfu DNA polymerase, Vent DNA polymerase, Tli DNA polymerase, Pwo DNA polymerase, and a blend of enzymes thereof.

9. (canceled)

10. (canceled)

11. (canceled)

12. (canceled)

13. (canceled)


SEQUENCE LISTING (8 sequences)

SEQ ID NO: 1 - DNA, 20 bp, Artificial Sequence
  forward primer for beta-globin
gaaggctcatggcaagaaag

SEQ ID NO: 2 - DNA, 20 bp, Artificial Sequence
  reverse primer for beta-globin
gattccgggtcactgtgagt

SEQ ID NO: 3 - DNA, 18 bp, Artificial Sequence
  forward primer for human retinoblastoma 1
caggacagcggcccggag

SEQ ID NO: 4 - DNA, 18 bp, Artificial Sequence
  reverse primer for human retinoblastoma 1
ctgcagacgctccgccgt

SEQ ID NO: 5 - DNA, 22 bp, Artificial Sequence
  forward primer for c-jun
gggaggggaccggggaacagag

SEQ ID NO: 6 - DNA, 22 bp, Artificial Sequence
  reverse primer for c-jun
gaacagtccgtcacttcacgtg

SEQ ID NO: 7 - DNA, 20 bp, Artificial Sequence
  forward primer for p53
tgccgtcccaagcaatggat

SEQ ID NO: 8 - DNA, 20 bp, Artificial Sequence
  reverse primer for p53
tgtgcagggtggcaagtggc